(19) United States
(12) Patent Application Publication
(10) Pub. No.: US 2026/0335500 A1
(43) Pub. Date: Oct. 29, 2026

(54) PASTY, BIOCOMPATIBLE MATERIAL FOR USE IN A METHOD FOR SUPPORTING BONE REGENERATION, THE COMPOSITION THEREOF, AND MOULDED PART FORMED FROM THE PASTY MATERIAL

(71) Applicant: Antonis Alexakis, Oehningen (DE)

(72) Inventor: Antonis Alexakis, Oehningen (DE)

(21) Appl. No.: 18/993,389

(22) PCT Filed: Jul. 10, 2023
(86) PCT No.: PCT/EP2023/069087

§ 371 Date: Jan. 10, 2025

(30) Foreign Application Priority Data
Jul. 11, 2022 (DE) 10 2022 117 264.3

(51) Int. Cl. A61L 27/02; A61L 27/36; A61L 27/54

(52) U.S. Cl. CPC A61L 27/025; A61L 27/3608; A61L 27/54; A61L 2300/406; A61L 2400/06; A61L 2430/02

(57) ABSTRACT

A pasty, biocompatible material for use in a method for supporting bone regeneration in a planar lesion not determined by surrounding bone material, on a bone base or in or on a bone, in a jaw bone, wherein the pasty biocompatible material is designed, after filling in the planar lesions not determined by surrounding bone material, to cure and to form a dimensionally stable molded part, wherein the material consists of or comprises between 50 wt. % and 70 wt. %, preferably 65 wt. %, of a calcium-containing base material, between 15 wt. % and 40 wt. %, preferably 20 wt. %, mineral trioxide aggregate and between 15 wt. % and 25 wt. % of at least one antibiotic, between 0.1 wt. % and 1.0 wt. % of a liquid, demineralized water or sodium chloride solution (NaCl), and the base material is completely resorbed once regeneration of bone material and/or bone augmentation is complete.

### FIELD

The present invention relates to a pasty, biocompatible material for supporting new bone formation in planar lesions which are not determined by surrounding bone material on or in a bone, in particular a jawbone. In this context, the situation is to be understood as planar when bone material has been resorbed in such a way that only the bone base is present, but not surrounding bone material or bone walls. The latter is the case, for example, with a tooth extraction. The cavity created by the extraction is surrounded on four sides by bone material that provides bone cells for independent regeneration and new bone formation.

The material consists of a combination of calcium-containing base material, mineral trioxide aggregate (MTA), antibiotic and a liquid. The calcium-containing base material is completely resorbed, while the MTA remains in the newly formed bone.

### BACKGROUND

Various materials have been used to date for bone repair and regeneration. However, many of these materials have the disadvantage of insufficient biocompatibility or insufficient stability and strength. Also, known materials are resorbed too quickly and therefore cannot be used for satisfying bone augmentation. Known uses of MTA only show treatment success in minor, clearly defined lesions surrounded on several sides by bone material.

In dentistry in particular, the known methods prove to be disadvantageous. When treating bone loss caused by periodontitis, the aim is to fill planar lesions without surrounding bone material with new bone material. Known replacement materials have the disadvantage that they are resorbed too quickly, so that new bone is not formed to a sufficient extent. The newly formed bone material must, however, be sufficiently stable and available in a quantity and height adapted to the original bone in order to be able to for example anchor implants stably in a follow-up treatment.

Until now, replacement parts made from the patient's own bone material, taken from the jaw joint or hip bone, have also been used in the treatment of periodontosis.

However, the associated procedures are complex and very stressful for the patient, and they are not always successful.

### SUMMARY

The present invention overcomes the disadvantages of known materials and treatments by providing a pasty, biocompatible material that has been developed specifically for new bone formation in planar lesions not determined by surrounding bone material. The material hardens after the filling of the lesions and forms a dimensionally stable molded part therein. The pasty material consists of a mixture of calcium-containing base material, mineral trioxide aggregate (MTA), antibiotic and a liquid. In this context, the situation is considered to be planar if bone material has been resorbed in such a way that only the bone base is present, but not surrounding bone material or bone walls. The latter is the case, for example, with a tooth extraction. The cavity created by the extraction is surrounded by bone material on four sides, which provides bone cells for autogenous regeneration and new bone formation. The dimensionally stable molded part that is formed allows the migration of bone cells from the base of the bone and provides a stable base and scaffold structure for the formation of new bone and the complete regeneration of the lesion, as well as sufficient bone augmentation.

The calcium-bearing base material, which constitutes between 50% and 70% by weight, preferably 65% by weight, of the material, can originate from a plurality of sources, including aragonite, mussel shell, shell lime, allogenic bone material, autogenic bone material, xenogenic bone material, FDBA (freeze-dried bone allografts), DFBA (decalcified freeze-dried bone allografts), algae or algae extract, ceramic, calcium phosphate, in particular tricalcium phosphate or tetracalcium phosphate, X- or β-tricalcium phosphate, hydroxyapatite, calcium phosphate ceramic, bioglass, aragonite-based bone replacement material (e.g. BioCoral®) or mixtures thereof.

In particular, it is also conceivable to produce the calcium-containing base material from donor bones. Calcium-containing base material produced from bones in bone banks is also encompassed by the invention.

The invention also considers the use of FDBA (freeze dried bone allografts) or DFDBA (decalcified freeze dried bone allografts) to be advantageous. By forming the calcium-containing base material from a material retrieved from a genetically different individual of the same species, bone growth can proceed optimally. The probability of inflammatory reactions is advantageously reduced. The use of xenogenic materials for the production of the calcium-containing base material has also proven to be favorable. For the production of calcium-containing base materials that are suitable for humans, bones from cattle, pigs and horses are particularly suitable. It is also possible, and encompassed by the invention, to provide the calcium-containing base material from algae, in particular algae extracts, corals or mussels. The shells of mussels have proved to be particularly suitable for the production of the calcium-containing base material, since they consist of a calcium-protein mixture, more precisely of aragonite, and can therefore be particularly well resorbed by the body.

In addition, it is also possible to produce the calcium-containing base material from autogenous material, i.e. material provided by the patient himself. For this purpose, bone material is first retrieved from the patient, processed into powder or granular material and prepared for use in the pasty material according to the invention, which is inserted or implanted in the patient in the course of further treatment. Thereby, the probability of inflammatory reactions occurring in the patient's body is lowest. Furthermore, it is possible to use alloplastic materials such as calcium phosphates, ceramics or bioglasses for the production of the calcium-containing base material. The mineral trioxide aggregate (MTA), which constitutes up between 15% and 40% by weight of the material, preferably 20% by weight, consists of a mixture of tricalcium silicate, dicalcium silicate, tricalcium aluminate and gypsum.

In a preferred further development, the mineral trioxide aggregate (MTA) can additionally contain a substance that increases radio-opacity, such as bismuth(III) oxide. The portion is then between 0.1 and 30% by weight, preferably 20% by weight of bismuth(III) oxide (Bi₂O₃) in the MTA component.

The antibiotic, which constitutes between 10% and 25% by weight of the material, can be selected from a plurality of antibiotics used in particular in dentistry or mixtures of several antibiotics.

The pasty, biocompatible material may also additionally contain a further substance that supports new bone formation. This further substance can be selected from the group of statins, vitamin, trace element, hyaluronic acid, hyaluronic acid derivative, collagen and/or mixtures thereof. The portion of this additional substance in the material is preferably between 0.1% and 3% by weight, in particular between 0.2% and 1.5% by weight, preferably 0.25% by weight.

The hyaluronic acid (or hyaluronic acid derivatives) that can be used in connection with the present invention has a favorable effect on the treatment of pathological changes of the periodontium and has positive effects on fibroblasts, bone regeneration and wound healing. In connection with the present invention, hyaluronic acid (respectively its derivatives) can be added or admixed directly in the composition according to the invention. Alternatively, after preparation of the pasty material and during the insertion or application on a bone base, the addition or irrigation of the site of application can be carried out with
a hyaluronic acid preparation. Hyaluronic acid has various functions in this process. The basic principle of action of hyaluronic acid in connection with the present invention is that in an aqueous environment, as a result of a spontaneous aggregation of the hyaluronic acid chains, three-dimensional mesh networks are formed. Cellular and fibrous components can be embedded in these. This favors and promotes the formation of a bone structure. At the same time, hyaluronic acid has a regulatory function in the organization of the extracellular matrix and its components. The hyaluronic acid network that is formed is a prerequisite for the exchange of substances and at the same time serves as a barrier against the penetration of exogenous substances. By forming and condensing these networks, cells can be protected from degradation processes and hydroxyl radicals. The resulting hyaluronic acid sheaths serve to protect various cell types from external, e.g. viral or bacterial, influences and thus also enhance the osteoblasts' probability of survival. Negatively charged hyaluronic acid is also capable of binding large amounts of water and various plasma proteins via hydrogen bonds and the polar ends, thus acting as a kind of “osmotic buffer” for the extracellular matrix. Hyaluronic acid also proves beneficial in combating chronic inflammation and has an anti-inflammatory potential. Hyaluronic acid also influences cellular growth factors and thus has a positive impact on cellular growth processes, supporting tissue regeneration. These numerous advantages are utilized in connection with the present invention and composition.

Surprisingly, it has been shown that the regeneration of bone or bone material can be significantly improved. Surprisingly, it has been shown that the present invention enables a form of ossification or osteogenesis that is clearly superior to the prior art, in particular in patients in which bone material has been resorbed in such a way that only the bone base is remaining, but not the surrounding bone material or bone walls. In contrast to this, for example, in cavity-creating treatments such as tooth extractions, the cavity created by the extraction is surrounded by bone material on four sides. This provides bone cells for autogenous regeneration and new bone formation. However, in the case of planar lesions, spontaneous bone regeneration and augmentation from the bone base is not possible.

With respect to the other optional additives selected from the group consisting of statins, vitamins, trace elements or mixtures thereof, vitamins and trace elements enhance the supply of the newly formed cells, while statins or statin preparations serve to modulate the immune system and thus reduce the tendency to inflammation. The invention is not limited to the substances mentioned hereinbefore, but includes all substances and substance mixtures which are known to those skilled in the art and can be used in connection with the present invention.

The mineral trioxide aggregate (MTA) in the pasty material consists in particular of a mixture of tricalcium silicate, dicalcium silicate, tricalcium aluminate and gypsum. The portion of a mixture of tricalcium silicate (CaO3·SiO2), dicalcium silicate (CaO₂·SiO₂), tricalcium aluminate (CaO₃·Al₂O₃), in the MTA is preferably between 70 and 80% by weight, preferably 75% by weight, while the portion of gypsum (CaSO₄·2H₂O) is between 1 and 10% by weight, preferably 5% by weight.

The pasty, biocompatible material is used to form a dimensionally stable molded part after curing. This molded part is formed by applying the pasty material to the remaining bone base in a planar lesion that is not determined by surrounding bone material under the periosteum and then curing it. The periosteum covers the lesion and is preferably under-injected with the pasty material.

The pasty, biocompatible material for supporting new bone formation and/or for bone augmentation consists of 50% by weight to 70% by weight of a calcium-containing base material, 15% by weight to 40% by weight of mineral trioxide aggregate (MTA), 15% by weight to 25% by weight of one or more antibiotics and 0.1% by weight to 1.0% by weight of a liquid, which is preferably demineralized water or sodium chloride solution (NaCl). The calcium-containing base material can originate from various sources, such as aragonite, mussel shell, allogenic bone material, autogenic bone material, xenogenic bone material, FDBA (freeze-dried bone allografts), DFBA (decalcified freeze-dried bone allografts), algae or algae extract, ceramic, calcium phosphate (in particular tricalcium phosphate or tetracalcium phosphate, X- or tricalcium phosphate, hydroxylapatite), calcium phosphate ceramic, bioglass, aragonite-based bone replacement material (e.g. BioCoral®) or mixtures thereof. The antibiotic can be selected from a group of antibiotics used particularly in dentistry or mixtures thereof. Exemplary antibiotics are penicillin, amoxicillin, clindamycin, metronidazole, erythromycin, tetracycline, doxycycline, ciprofloxacin, levofloxacin, azithromycin, minocycline, lincomycin, gentamicin, vancomycin, moxifloxacin, rifampicin, sulfamethoxazole/trimethoprim, amikacin, ceftazidime, ceftriaxone, imipenem, meropenem, ampicillin, chloramphenicol, nystatin, ketoconazole, fluconazole or voriconazole, without limiting the invention thereto.

Surprisingly, it has been shown that the non-resorbable MTA in the biocompatible molded part has several beneficial effects on new bone formation, thus enabling a significantly higher growth of bone material. In the course of new bone formation, in particular in the jawbone, the calcium-containing base material is resorbed, but the non-resorbable mineral trioxide aggregate (MTA) remains in the bone material newly formed in the filled bone lesion and is incorporated into the newly forming bone substance. During the resorption of the calcium-containing base material, mineral trioxide aggregate (MTA) forms a scaffold structure. Osteoclasts decompose the calcium-containing base material during the process of new bone formation, thus providing a foundation for the deposition of osteoblasts in the cavities formed by the decomposition. The bone structure surrounding the cavities is further stabilized by mineral trioxide aggregate (MTA), thus extending the period during which bone growth is possible. This results in a larger, i.e. higher, bone augmentation and enables the formation of new bone in larger bone sections or lesions, respectively. This also applies to patients in whom only the bone base is remaining. Mineral trioxide aggregate (MTA) also provides a basis for cell growth, as bone cells aggregate on the mineral trioxide aggregate (MTA), thus additionally enhancing new bone formation. The bone augmentation of an existing bone structure is significantly improved. In particular, bone augmentation of up to between 1.8 and 2.0 cm is possible. Mineral Trioxide Aggregate (MTA) has the added benefit of a blood-binding effect directly after the surgical procedure and also has an anti-inflammatory effect due to the alkaline pH value in the surgical area.

During use, i.e. in the course of bone formation, the molded part formed from the hardened pasty material in the lesion is resorbed except for the mineral trioxide aggregate (MTA) portion. This resorption is accompanied by a corresponding filling of the lesion with newly formed bone material or the filling with osteoblasts, which initiate or carry out osteogenesis.

This invention thus provides an improved pasty biocompatible material for supporting new bone formation and bone augmentation, which has been specially developed for the treatment of lesions on or in bones, in particular jawbones, in which only the bone base is present, but no surrounding bone material or bone walls.

The present invention also relates to a composition for producing a pasty biocompatible material for use in planar lesions on or in a bone, in particular in a jawbone, which are not determined by surrounding bone material. The composition consists of a calcium-containing, structure-providing base material, mineral trioxide aggregate (MTA) and at least one antibiotic.

The calcium-containing structuring material can be selected from a plurality of materials, including aragonite, mussel shell, allogenic bone material, autogenic bone material, xenogenic bone material, FDBA (freeze-dried bone allografts), DFBA (decalcified freeze-dried bone allografts), algae or algae extract, ceramic, calcium phosphate (in particular tricalcium phosphate or tetracalcium phosphate, X- or β-tricalcium phosphate, hydroxylapatite), calcium phosphate ceramic, bioglass, aragonite-based bone replacement material (e.g. BioCoral®) or mixtures thereof.

The mineral trioxide aggregate (MTA) in the composition consists of tricalcium silicate (CaO₃·SiO₂), dicalcium silicate (CaO₂·SiO₂), tricalcium aluminate (CaO₃·Al₂O₃) and gypsum (CaSO₄·2H₂O) or comprises the same.

In a preferred embodiment, the composition of the mineral trioxide aggregate (MTA) additionally contains a radio-opacity enhancing substance, in particular bismuth(III) oxide (Bi₂O₃), in an amount between 0.1 and 30% by weight, preferably 20% by weight.

The composition also contains at least one antibiotic selected from a group of antibiotics that are particularly suitable for use in dentistry, preferably in bone regeneration.

The composition typically consists of 50% by weight to 70% by weight, preferably 65% by weight of the calcium-containing base material, 15% by weight to 40%, preferably 20% of the mineral trioxide aggregate (MTA), and 15% to 25% of the antibiotic.

The mineral trioxide aggregate (MTA) is provided in the form of a powder or as granular material and is homogeneously dispersed in the base material. These properties allow for easy handling and use of the composition.

The composition according to the invention provides an improved treatment option for bone lesions, particularly in the jawbone, by supporting tissue regeneration and wound healing and creating a suitable environment for cells.

To produce the pasty biocompatible material, the previously described components in powder form (calcium-containing structure-providing base material, mineral trioxide aggregate (MTA) and antibiotic(s) as well as optionally further substances mentioned hereinbefore) are first mixed and then, after adding the liquid, preferably demineralized water or sodium chloride solution (NaCl), are stirred to the desired consistency. The pasty material can then be drawn up with a syringe and introduced into the lesion.

The present invention also relates to a biocompatible molded part for use in a method of supporting new bone formation, in particular for bone augmentation of an existing bone structure. The biocompatible molded part is characterized in that it is formed from a pasty material as described above after a planar lesion on or in a bone, in particular in a jawbone, that is not determined by surrounding bone material, has been filled with the pasty material. The molded part consists of a calcium-containing, structure-providing base material, mineral trioxide aggregate (MTA), and at least one antibiotic.

It is considered advantageous if the molded part contains between 50% by weight and 70% by weight, preferably 65% by weight, of the calcium-containing base material, between 15% by weight and 40% by weight, preferably 20% by weight, of the mineral trioxide aggregate (MTA), and between 15% by weight and 25% by weight of at least one antibiotic.

In addition, the molded part may contain, in particular, at least one further substance. The further substance can be selected from the group consisting of statin, vitamin, trace element, hyaluronic acid, hyaluronic acid derivative, collagen and/or mixtures thereof. The portion of the further substance in the molded part is in particular between 0.1% by weight and 3% by weight, preferably between 0.2% by weight and 1.5% by weight, wherein 0.25% by weight is preferred.

When used in a method of supporting new bone formation, particularly in a jawbone, the molded part provides a basic structure for new bone formation. The base material and the antibiotic are completely resorbed in the course of the new bone formation, while the mineral trioxide aggregate (MTA) remains in the newly formed bone. The molded part allows the filling of planar lesions on or in a bone, in particular in a jawbone, which are not clearly defined by surrounding bone material or where only the bone base is present, but not surrounding bone material or bone walls.

Therein, the molded part is preferably formed in situ in a planar lesion on or in a bone, which is not determined by surrounding bone material, from a hardened, pasty material. In a preferred embodiment, the molded part is formed after under-injection of a periosteum covering the planar lesion not determined by surrounding bone material, directly in the lesion and thus formed adapted to its shape and extent.

The present invention enables effective support of new bone formation and bone augmentation in existing bone structures. The biocompatible molded part provides a structure-providing base structure and is resorbed in the course of new bone formation, while the mineral trioxide aggregate (MTA) remains in the bone. Additional advantages can be achieved by adding further substances.

The invention also relates to mineral trioxide aggregate (MTA) for use in the treatment of bone deficits, in particular in the treatment of periodontal disease in a biocompatible molded part formed from a pasty material, wherein the filling of a planar lesion on or in a bone, in particular in a jawbone, that is not determined by surrounding bone material is provided.

The present invention relates to a mineral trioxide aggregate (MTA), which was developed specifically for the treatment of bone deficiencies, in particular for the treatment of periodontal disease. Periodontal disease is a condition in which there is a loss of bone tissue around the teeth, leading to a weakening of the periodontium. Various methods are used to regenerate bone in order to counteract these bone deficiencies.

Mineral trioxide aggregate (MTA) has been used in dentistry, particularly in bacteria-proof, retrograde root canal sealing in apical surgery.

Surprisingly, it has been shown that mineral trioxide aggregate (MTA) enhances new bone formation in planar lesions on or in a bone, especially in a jawbone, that are not clearly determined by surrounding bone material. In such planar lesions, the migration of bone cells into the treatment area is more difficult because, unlike in cavities that need to be filled, the surrounding bone tissue is missing and the migration can only occur from one side/surface.

Mineral trioxide aggregate (MTA) significantly enhances the treatment of bone defects, for example those resulting from periodontitis. Due to the original use of MTA in dental therapy as a sealing material for root canals and perforations in milk teeth or for filling cavities, the use of mineral trioxide aggregate (MTA) in the treatment of planar lesions or bone deficiencies on or in a bone, especially in a jawbone, which are not clearly determined by surrounding bone material, by new formation of bone material to fill defects in bones after surgery or disease-related bone loss, or the formation of bone lesions where filling with newly formed bone material is indicated, and in particular in the treatment of periodontitis, is new and not obvious to a person skilled in the art.

Mineral trioxide aggregate (MTA) is known from its use in the field of oral surgery and is suitable for use therein. However, the use of a pasty, biocompatible material, as described above, and a molded part that can be formed from it, as per the present invention, is not previously known and has surprisingly led to a significant improvement in new bone formation, particularly in the treatment of periodontitis-related bone damage. Due to the lack of resorption, Mineral Trioxide Aggregate (MTA) provides the molded part with good stability of the molded part or solid body over a long period of time during bone growth.

At the same time, mineral trioxide aggregate (MTA) enhances cell growth and vascular ingrowth. Mineral trioxide aggregate (MTA) is also able to bind and stabilize blood coagulates that form. This increases the chances that vessels can grow throughout the lesion at the same time. These vessels serve to supply the osteoblasts with nutrients, so that ossification or osteogenesis can occur, in the course of which new bone material is formed and thus, for example, a jawbone that has been pre-damaged by periodontitis can be rebuilt.

The molded part, which is preferably formed in situ, is more dimensionally stable over a longer period of time than known molded parts and can be individually dimensioned so that after sufficient ossification or osteogenesis, i.e. when no supporting structure is needed, the resorption process is complete and mineral trioxide aggregate (MTA) continues to remain in the bone as a supporting matrix.

According to the present invention, mineral trioxide aggregate (MTA) is used in a pasty material that is molded into a biocompatible part. This molded part allows for the precise placement of the mineral trioxide aggregate (MTA) at the affected sites, especially in the case of planar lesions that are not determined by surrounding bone material. This is particularly advantageous in the treatment of periodontal disease, as it allows for targeted and effective filling of the bone deficiencies.

The use of the mineral trioxide aggregate (MTA) in a biocompatible molded part offers several advantages. First, it enables improved adhesion and fixation of the mineral trioxide aggregate (MTA) at the target sites, ensuring long-term stability and effectiveness. Second, the biocompatible material of the molded part ensures minimal irritation and good compatibility with the surrounding tissue, resulting in a lower inflammatory response and faster recovery.

The use of the mineral trioxide aggregate (MTA) and the biocompatible molded part containing it is particularly preferred in the treatment of periodontal disease in the jawbone. The jawbone is prone to bone deficiencies due to periodontal disease, and the targeted use of the mineral trioxide aggregate (MTA) in connection with the biocompatible molded part can result in effective regeneration of the bone.

Overall, the present invention offers an improved solution for the treatment of bone deficiencies.

The beneficial effect of the material according to the invention is also confirmed by clinical studies.

In a patient, the pasty material according to the invention was implanted under the periosteum into a region with degraded jawbone after local anesthesia, and the biocompatible molded part was formed in situ therein.

The pasty material was produced by mixing
60% by weight shell lime,
20% by weight mineral trioxide aggregate (MTA) and
20% by weight of an antibiotic, each in the form of a powder. A few drops of a sodium chloride solution (NaCl) were added,
   drawn up using a conventional cannula and then injected under the periosteum to the desired later height of the bone augmentation.

Six months after treatment, a bone cylinder with a maximum length of 0.7 cm and diameter of 0.2 cm was retrieved from the region in which new bone had formed, and the regeneration potential of the material was determined on the basis of this sample. Thin sections were prepared for histology and microscopically examined.

Microscopic evaluation showed that vital corticospongiosa bone had formed with focal evidence of osteoblast seams and relatively fresh newly formed portions. Focal, sparse and peripheral amorphous material and extremely sparse minor avital necrotic bone fragments were identifiable.

The result showed predominantly vital, partly fresh corticospongiosa bone with signs of growth and medullary fibrosis. Foreign body reactions and amorphous material occurred focally in the connective tissue. Overall, however, at most microfocal evidence of non-vital necrotic bone or bone replacement material was detectable. However, vital bone with fresh portions and new bone formation had formed predominantly as a sign of regeneration. There was no evidence of florid osteomyelitis and no evidence of malignancy.

Five other patients examined showed the same findings.

### BRIEF DESCRIPTION OF THE DRAWINGS

Further advantages and functional designs can be retrieved from the description of the figures and the figures. It shows:

FIG. 1 a schematic representation of the situation of a lesion before treatment,

FIG. 2 a schematic representation of the situation after the lesion has been filled and the biocompatible molded part according to the invention has formed,

FIG. 3 a schematic representation of the situation with a completely regenerated lesion and bone augmentation.

### DETAILED DESCRIPTION

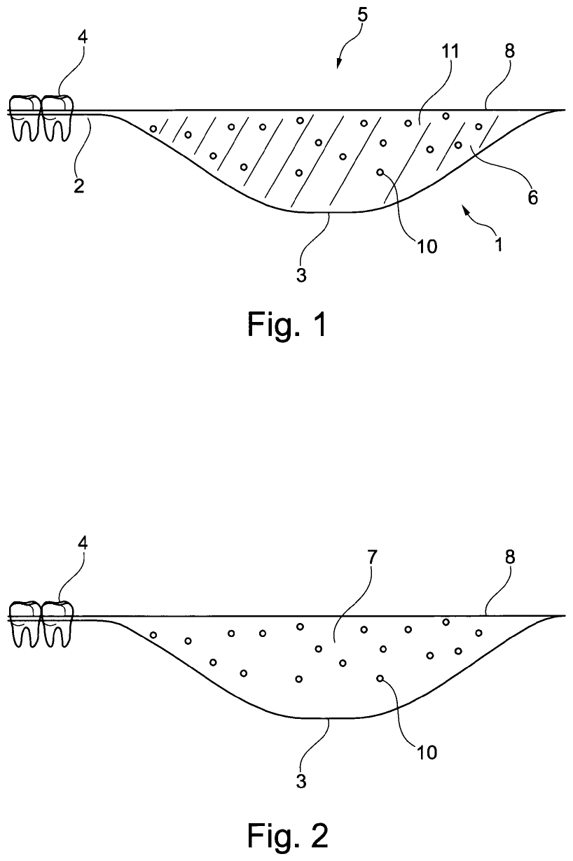

### FIG. 1: Situation of a Lesion Before Treatment

FIG. 1 schematically shows the situation of a lesion 1 immediately after treatment with the pasty material of the invention. It consisted of a planar lesion 1, in which the surrounding bone material 2 was resorbed and only the bone base 3 is still present. This can occur, for example, in periodontitis in the jawbone. FIG. 1 shows the remaining teeth 4 outside the degraded bone structure. Unlike the situation after tooth extraction, the space 5 is not surrounded by bone material that could provide regeneration and new formation of bone cells. The lesion 1 is covered by the periosteum 8.

FIG. 2 shows a schematic illustration of the situation after filling lesion 1 with the pasty, biocompatible material 6. The material 6 has hardened and formed a dimensionally stable molded part 7 in the lesion 1. This creates a stable base and scaffold structure that enables the bone cells to penetrate the lesion 1 and form new bone. In FIG. 2, the molded part 7 was formed in situ in a planar lesion 1, not determined by surrounding bone material, on the remaining bone base 3 from the pasty material of the present invention and after its hardening. The molded part 7 is formed directly in the lesion 1 and thus adapted to the shape and extent of the lesion, after under-injection of a periosteum 8 covering the lesion, on the bone base 3.

The non-resorbable MTA 10 in the biocompatible molded part 7 enables a significantly higher growth of bone material. During the process of new bone formation, particularly in the jawbone, the calcium-containing base material is resorbed, but the non-resorbable mineral trioxide aggregate (MTA) remains in the new bone material formed in the filled lesion and is incorporated into the newly forming bone substance. During the resorption of the calcium-containing base material, mineral trioxide aggregate (MTA) forms a scaffold structure. Osteoclasts degrade the calcium-containing base material during the process of new bone formation, thus providing the basis for the colonization of osteoblasts in the cavities formed by the degradation.

The bone structure surrounding the cavities is additionally stabilized by mineral trioxide aggregate (MTA) and the period during which bone growth is possible is thus extended. This results in a larger, i.e. higher, bone augmentation or enables the formation of new bone in larger bone sections or lesions 1. This also applies to patients in whom, as shown in FIG. 1, only the bone base 3 is remaining. Mineral Trioxide Aggregate (MTA) also provides a basis for cell growth, as an attachment of bone cells to the Mineral Trioxide Aggregate (MTA) occurs, thus further enhancing new bone formation. The bone augmentation of an existing bone structure is significantly improved. In particular, bone augmentation of up to between 1.8 and 2.0 cm is possible. Mineral Trioxide Aggregate (MTA) exhibits a blood-binding effect directly after the surgical procedure as a further advantage and also has an anti-inflammatory effect due to the alkaline pH value in the surgical area.

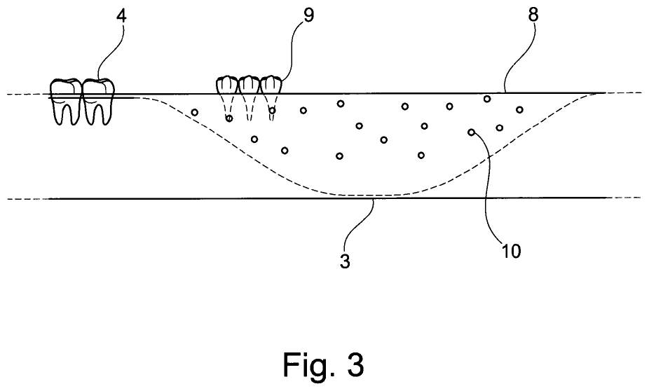

FIG. 3 schematically shows the situation after complete regeneration of lesion 1 and bone augmentation. The pasty material has led to the formation of new bone that adjoins existing bone material and then raises the bone base 3, which has completely regenerated the lesion 1. As a result, there is a sufficient amount and height of bone, for example, to stably anchor implants 9.

1. A pasty, biocompatible material for use in a method of supporting new bone formation in a planar lesion not determined by surrounding bone material, on a bone base or in or on a bone, wherein the pasty, biocompatible material is designed to harden after filling the planar lesions not determined by surrounding bone material and to form a dimensionally stable molded part, wherein the material comprises:
between 50% by weight and 70% by weight of a calcium-containing base material,
between 15% by weight and 40% by weight of mineral trioxide aggregate, and
between 15% by weight and 25% by weight of at least one antibiotic,
between 0.1% by weight and 1.0% by weight of a liquid, water or sodium chloride solution
and wherein the base material is completely resorbed by the time new bone material has formed and/or bone augmentation has been completed.

2. The pasty, biocompatible material according to claim 1, wherein
the base material is selected from the group consisting of: aragonite, mussel shell, shell lime, allogenic bone material, autogenic bone material, xenogenic bone material, FDBA (freeze-dried bone allografts), DFBA (decalcified freeze-dried bone allografts), algae or algae extract, ceramic, calcium phosphate, tricalcium phosphate or tetracalcium phosphate, x- or β-tricalcium phosphate, hydroxylapatite, calcium phosphate ceramic, bioglass, aragonite-based bone replacement material or mixtures thereof,
the mineral trioxide aggregate (MTA) comprises or consists of tricalcium silicate (CaO₃·SiO₂), dicalcium silicate (CaO₂·SiO₂), tricalcium aluminate (CaO₃·Al₂O₃) and gypsum (CaSO₄·2H₂O), and
the antibiotic is selected from the group consisting of penicillin, amoxicillin, clindamycin, metronidazole, erythromycin, tetracycline, doxycycline, ciprofloxacin, levofloxacin, azithromycin, minocycline, lincomycin, gentamicin, vancomycin, moxifloxacin, rifampicin, sulfamethoxazole/trimethoprim, amikacin, ceftazidime, ceftriaxone, imipenem, meropenem, ampicillin, chloramphenicol, nystatin, ketoconazole, fluconazole or voriconazole or mixtures thereof.

3. The pasty, biocompatible material according to claim 1, wherein the material contains at least one further substance, wherein the at least one further substance is selected from the group consisting of statin, vitamin, trace element, hyaluronic acid, hyaluronic acid derivative, collagen and/or mixtures thereof, wherein the at least one further substance has a portion of at least one of: i) between 0.1-3% by weight of the pasty, biocompatible material; ii) between 0.2-1.5% by weight of the pasty, biocompatible material; or iii) 0.25% by weight of the pasty, biocompatible material.

4. The pasty, biocompatible material according to claim 1, wherein the mineral trioxide aggregate contains
at least one of: i) between 70 and 80% by weight of a mixture of tricalcium silicate (CaO₃·SiO₂), dicalcium silicate (CaO₂·SiO₂), tricalcium aluminate (CaO₃·Al₂O₃), or ii) 75% by weight of the mixture of tricalcium silicate (CaO₃·SiO₂), dicalcium silicate (CaO₂·SiO₂), tricalcium aluminate (CaO₃·Al₂O₃), and
at least one of: i) between 1 and 10% by weight of gypsum (CaSO₄·2H₂O), or ii) 5% by weight of gypsum (CaSO₄·2H₂O).

5. The pasty, biocompatible material according claim 1, wherein the mineral trioxide aggregate contains a radio-opacity enhancing substance, the radio-opacity enhancing substance comprising bismuth(III) oxide (Bi₂O₃).

6. The pasty, biocompatible material according to claim 5, wherein the mineral trioxide aggregate contains i) between 0.1 and 30% by weight of bismuth(III) oxide (Bi₂O₃), or ii) preferably 20% by weight of bismuth(III) oxide (Bi₂O₃).

7. The pasty, biocompatible material according to claim 1, wherein the dimensionally stable molded part is formed after injection of a periosteum covering the planar lesion not determined by surrounding bone material after hardening of the pasty, biocompatible material.

8. A pasty, biocompatible material for supporting the formation of new bone material and/or for bone augmentation in a mammalian bone by forming a molded part after hardening, wherein the pasty biocompatible material comprises:
at least one of: i) between 50% by weight and 70% by weight of a calcium-containing base material, or ii) 65% by weight, of a the calcium-containing base material,
at least one of: i) between 15% by weight and 40% by weight of mineral trioxide aggregate (MTA); or ii) 20% by weight of mineral trioxide aggregate (MTA),
between 15% by weight and 25% by weight of at least one antibiotic, and
between 0.1% by weight and 1.0% by weight of a liquid, the liquid comprising one of demineralized water or sodium chloride solution wherein
the calcium-containing base material is selected from the group consisting of: aragonite, mussel shell, shell lime, allogenic bone material, autogenic bone material, xenogenic bone material, FDBA, DFBA, algae or algae extract, ceramic, calcium phosphate, tricalcium phosphate or tetracalcium phosphate, x- or β-tricalcium phosphate, hydroxylapatite, calcium phosphate ceramic, bioglass, aragonite-based bone replacement material or mixtures thereof,
the mineral trioxide aggregate (MTA) comprises or consists of tricalcium silicate (CaO₃·SiO₂), dicalcium silicate (CaO₂·SiO₂), tricalcium aluminate (CaO₃·Al₂O₃) and gypsum (CaSO₄·2H₂O), and
the antibiotic is selected from the group consisting of penicillin, amoxicillin, clindamycin, metronidazole, erythromycin, tetracycline, doxycycline, ciprofloxacin, levofloxacin, azithromycin, minocycline, lincomycin, gentamicin, vancomycin, moxifloxacin, rifampicin, sulfamethoxazole/trimethoprim, amikacin, ceftazidime, ceftriaxone, imipenem, meropenem, ampicillin, chloramphenicol, nystatin, ketoconazole, fluconazole or voriconazole or mixtures thereof.

9. A composition for producing a pasty biocompatible material according to claim 1, for use in a planar lesion not determined by surrounding bone material on a bone base or on or in a bone in a jawbone, comprising or consisting of:
a calcium-containing structure-providing base material,
mineral trioxide aggregate, and
at least one antibiotic,
wherein
the calcium-containing structure-providing base material is selected from the group consisting of aragonite, mussel shell, shell lime, allogenic bone material, autogenic bone material, xenogenic bone material, FDBA, DFBA algae or algae extract, ceramic, calcium phosphate, tricalcium phosphate or tetracalcium phosphate, X- or β-tricalcium phosphate, hydroxylapatite, calcium phosphate ceramic, bioglass, aragonite-based bone graft substitute or mixtures thereof,
the mineral trioxide aggregate (MTA) comprises or consists of tricalcium silicate (CaO₃·SiO₂), dicalcium silicate (CaO₂·SiO₂), tricalcium aluminate (CaO₃·Al₂O₃) and gypsum (CaSO₄·2H₂O), and
the at least one antibiotic is selected from the group consisting of penicillin, amoxicillin, clindamycin, metronidazole, erythromycin, tetracycline, doxycycline, ciprofloxacin, levofloxacin, azithromycin, minocycline, lincomycin, gentamicin, vancomycin, moxifloxacin, rifampicin, sulfamethoxazole/trimethoprim, amikacin, ceftazidime, ceftriaxone, imipenem, meropenem, ampicillin, chloramphenicol, nystatin, ketoconazole, fluconazole or voriconazole, or mixtures thereof.

10. The composition according to claim 9, wherein the composition comprises or consists of:
at least one of: i) between 50% by weight and 70% by weight of the calcium-containing base material, or ii) 65% by weight of the calcium-containing base material,
at least one of: i) between 15% by weight and 40% by weight of the mineral trioxide aggregate, or ii) 20% by weight of the mineral trioxide aggregate (MTA), and
between 15% by weight and 25% by weight of the at least one antibiotic.

11. The composition according to claim 9, wherein mineral trioxide aggregate contains a radio-opacity enhancing substance comprising bismuth (III) oxide (Bi₂O₃).

12. The composition according to claim 11, wherein the mineral trioxide aggregate contains i) between 0.1 and 30% by weight of bismuth(III) oxide (Bi₂O₃), or ii) 20% by weight of bismuth(III) oxide (Bi₂O₃).

13. The composition according to claim 9, wherein i) the mineral trioxide aggregate (MTA) is provided in the form of a powder or as a granular material, and/or ii) the mineral trioxide aggregate (MTA) is homogeneously distributed in the base material.

14-15. (canceled)

16. A biocompatible molded part for use in a method of supporting new bone formation, wherein the biocompatible molded part is formed after filling a planar lesion, not determined by surrounding bone material, on a bone base or on or in a bone, from a pasty material according claim 1 and consists of a calcium-containing, structure-providing base material, mineral trioxide aggregate (MTA) and at least one antibiotic.

17. (canceled)

18. The biocompatible molded part according to claim 16, wherein the molded part contains at least one further substance, wherein the at least one further substance is selected from the group consisting of statin, vitamin, trace element, hyaluronic acid, hyaluronic acid derivative, collagen and/or mixtures thereof, wherein the at least one further substance preferably has a portion of at least one of: i) between 0.1-3% by weight of the molded part; ii) between 0.2-1.5% by weight of the molded part; or iii) 0.25% by weight of the molded part.

19. The biocompatible molded part according to claim 16, wherein the molded part, when used in a method of supporting new bone formation in a jawbone, provides a basic structure for the new bone formation, for the bone augmentation of an existing bone structure, and the base material and the antibiotic are completely resorbed in the course of the new bone formation and the mineral trioxide aggregate remains in the newly formed bone.

20. The biocompatible molded part according to claim 19, wherein the molded part is formed in situ in a planar lesion, not determined by surrounding bone material, on a bone base or on or in a bone, from a hardened, pasty material.

21. The biocompatible molded part according to claim 20, wherein the molded part is formed after injection under a periosteum covering the planar lesions not determined by surrounding bone material.

22. A mineral trioxide aggregate for use in the treatment of bone deficits, in the treatment of periodontal disease in a biocompatible molded part formed from the pasty material according to claim 1.

23. A mineral trioxide aggregate for use in the treatment of bone deficits, in the treatment of periodontal disease in a biocompatible molded part formed from the pasty material according to claim 1, wherein the filling of a planar lesion not determined by surrounding bone material on a bone base or on or in a bone, in a jawbone, is provided.
